(19) United States
(12) Patent Application Publication
(10) Pub. No.: US 2003/0091971 A1
(43) Pub. Date: May 15, 2003

(54) Composition for stabilizing biological materials

(75) Inventors: Jiu-Lin Xia, Germantown, MD (US); Sharon Challberg, Gaithersburg, MD (US); Fredric R. Bloom, Germantown, MD (US)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Invitrogen Corporation

(21) Appl. No.: 10/242,142

(22) Filed: Sep. 12, 2002

Related U.S. Application Data
(60) Provisional application No. 60/318,889, filed on Sep. 14, 2001.

(51) Int. Cl. A01N 1/02; C07H 21/04; C12N 9/00; C12N 1/00

(52) U.S. Cl. 435/2; 435/183; 435/317.1; 536/23.1

(57) ABSTRACT

A biomaterial is rendered storage stable by being incorporated into a water-soluble or swellable non-glass-forming composition which can be stored at ambient temperatures.

### BACKGROUND OF THE INVENTION

1. Field of the Invention

The present invention relates to non-glass-forming compositions for stabilizing biological materials, particularly enzymes and cells used in life science research and/or applications. The present invention particularly relates to compositions comprising a polar or partially charged polymer, or polyelectrolyte, and a biomaterial, and to the use of such compositions in a method for stabilizing biomaterials.

2. Brief Description of the Background Art

Biomolecules, especially proteins and polypeptide containing compounds, commonly exist in their naturally occurring hydrated state in the form of complex, three-dimensional folded confirmations generally known as tertiary structures. Very frequently, the activity of the compound, whether an enzyme, antibody, antigen, flavorant, fluorescent or gelling agent etc., is critically dependent on its tertiary structure and is severely reduced or even eliminated if its structure is disturbed, even though the chemical empirical formula of the compound may not have changed.

Macromolecular biological structures also depend upon specific molecular architectures to maintain their structural and/or functional integrity. For example, biological membranes are composed of lipid, protein and carbohydrate molecules, organized into a bilayer arrangement wherein the lipid molecules form the structural repeating units with protein or glycosylated protein molecules associated with or imbedded in the lipid matrix. As with biomolecules, if the membranes structural integrity is disrupted it loses its ability to function, that is to serve as a semi-permeable barrier.

A few biomaterials (e.g. some proteins) are sufficiently stable to be isolated, purified, and stored in solution at room temperature and still retain their activity. However, most are susceptible to degradation and therefore lose their desired activity after being stored for any significant period of time. The degradation of biomaterials, whether it's due to the loss of tertiary structure (denaturation) or some other process, is a function of a biomaterial's environment, and can be attributed to the sensitivities these substances have to the presence or absence of water and/or other organic or inorganic materials, and to pH and temperature extremes. The problems encountered with the manipulation and storage of these types of substances can be attributed to the aforementioned sensitivities, and have required the development of elaborate, work intensive procedures, for the storage and stabilization of these compositions. The following examples are illustrative of these procedures, and note their respective drawbacks.

(i) Addition of a high concentration of chemical “stabilizer” to the aqueous solution or suspension. Typically 3M ammonium sulphate is used. However, such additives can alter the measured activity of enzymes and can give ambiguous or misleading results if the enzyme is used in a test procedure. (R. H. M. Hatley and F. Franks. Variations in apparent enzyme activity in two-enzyme assay systems: Phosphoenolpyruvate carboxylase and malate dehydrogenase. Biotechnol Appl. Biochem. 11:367-370 (1989)). In the manufacture of diagnostic kits based on multi-enzyme assays, such additives often need to be removed before the final formulation. Such removal, by dialysis, often reduces the activity of an enzyme.

(ii) Freeze/thaw methods in which the preparation, usually mixed with an additive (referred to as cryoprotectant) is frozen and stored, usually below −50° C., sometimes in liquid nitrogen. Not all proteins will survive a freeze/thaw cycle.

(iii) Cold storage, with a cryoprotectant additive present in sufficient concentration (e.g. glycerol) to depress the freezing point to below the storage temperature and so avoid freezing. For example in the case of restriction endonucleases, the enzymes need to be protected against freezing by the addition of high concentrations of glycerol and maintained at −20° C. Use of an additive at a high concentration may also reduce the specificity of restriction enzymes and give rise to so-called “star-activity.” (B. Polisky et al., Proc. Natl. Acad. Sci. USA 72:3310 (1975)).

(iv) The most common method for the stabilization of isolated protein preparations is freeze-drying, but this process can only be applied to freeze-stable products. A suitable buffer containing the biological material is frozen, typically at −40° C. to −50° C. in the presence of a cryoprotectant, and then the ice is removed by sublimation under vacuum at low sub-zero temperatures. The residual moisture associated with the “dried” preparation, which may amount up to 50%, is then removed by desorption during which the temperature gradually rises. The complete freeze-drying cycle may take several days and is costly in capital and energy. Freeze-drying also suffers from technical disadvantages because of its irreproducibility. Suppliers of freeze-dried protein products generally specify storage at −20° C. rather than ambient temperature. Exposure to ambient temperatures for periods of days to weeks can result in significant activity losses.

(v) Undercooling, as described by Hatley et al. (Process Biochem. 22:169 (1987)) allows for the long-term (years) stabilization of proteins without the need for additives. However, while this process extended the previous repertoire of possibilities, the undercooled preparations need to be shipped at temperatures not exceeding +5° C. and must be stored refrigerated, preferably at −20° C. They also have to be recovered from a water-in-oil dispersion prior to their final use.

(vi) A body of work also exists on the effects of various glass-forming carbohydrates on the stabilization and storage of biomaterials. This process involves the incorporation of the biological substance to be stored into a carbohydrate glass-forming matrix. The vitrification of a biological substance can be accomplished by dissolving the substance to be stored in an aqueous solution containing a water-soluble or water swellable glass-forming substance, and removing the water to form the desired glass.

A glass, defined as an undercooled liquid with a high viscosity, is normally a homogeneous transparent brittle solid which can be ground or milled into a powder. Glasses are characterized by their glass-transition temperature (Tg), which is a sudden increase in heat capacity as diffusion becomes observable on the differential scanning calorimetry time scale (seconds) and liquid like degrees of freedom become accessible. This temperature designates at what point an amorphous glass-forming material changes from an elastic state to a plastic state. Above this temperature the viscosity drops rapidly and the glass turns into a rubber, then at even higher temperatures turns into a liquid. Below the Tg a glass's viscosity is extremely high, that is to say at least 10¹⁴ Pa.s or probably more. The absence of a Tg with the aforementioned characteristics indicates that a substance does not form a glass. The glass state results in the inhibition of molecular movement and extremely low rates of diffusion, such as microns per year, and chemical or biochemical reactions requiring the interaction of more than one reacting moiety are, for all practical purposes, completely inhibited, thereby suspending the decay process of any encased biological molecule.

However, if the biomaterial glass mixture is heated above its Tg during storage it will change to the rubbery state. The rubbery state does not afford an encased biomaterial the same protection as a glass. Therefore, if the rubbery state is maintained for any significant period of time the biomaterial is to prone to degradation.

Furthermore, if the Tg of the glass forming/biomaterial composition is close to or below room temperature it may be necessary to refrigerate the glassy composition in order to obtain the maximum protective effect.

Taking into consideration the state of the art concerning the preservation of biomaterials, it is apparent that an improved stabilization/storage process is needed. The present invention addresses this need by providing a method and composition for the storage and stabilization of biomaterials in a non-glass-forming substance.

### SUMMARY OF THE INVENTION

The present invention relates to a method and composition for the storage and/or stabilization of biomaterials in a non-glass-forming substance. The invention particularly relates to a water or buffer reconstitutable composition which is storage stable at ambient or refrigerated temperatures comprising:

i) a carrier substance which is a non-glass-forming water soluble or water swellable, natural or synthetic polar/partially charged polymer or polyelectrolyte and;

ii) one or more biomaterial(s) to be stored.

The invention also relates to a method of rendering a biomaterial storage stable at ambient or refrigerated temperatures comprising:

i) introducing a carrier substance which is a water-soluble or water-swellable natural or synthetic polar/partially charged polymer or polyelectrolyte into water or an appropriate buffer and;

ii) adding one or more biomaterial(s) to be stored;

iii) mixing the resulting suspension or solution and;

iv) removing the water to form the water or buffer reconstitutable composition.

### BRIEF DESCRIPTION OF THE DRAWINGS

FIG. 1 depicts the curve generated from a differential scanning calorimetry (DSC) of a sample of trehalose. The characteristic sudden increase in heat capacity at the transition temperature is apparent, and indicates that trehalose is a glass forming carbohydrate.

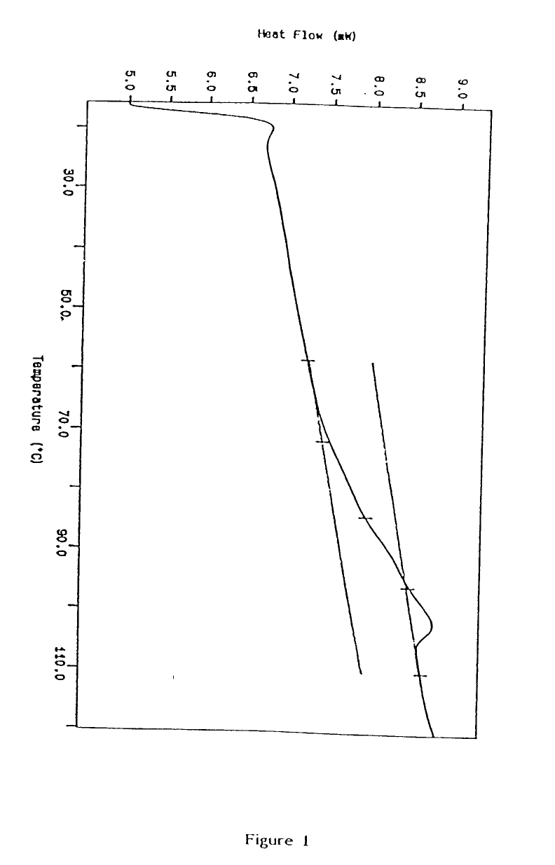

FIG. 2 depicts the curve generated from a DSC of a sample of “Ficoll.” The characteristic sudden increase in heat capacity is apparent, and indicates that Ficoll is a glass forming carbohydrate polymer.

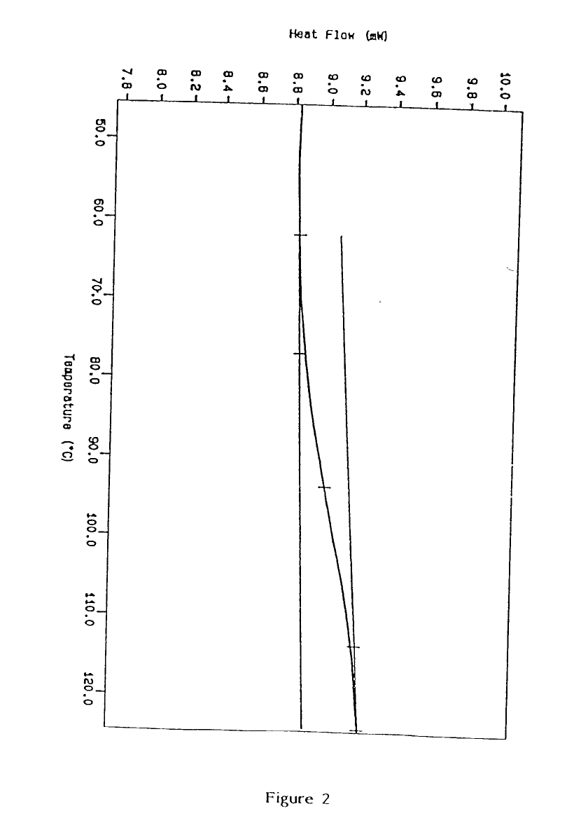

FIG. 3 depicts the DSC curve generated from a sample of acacia gum. This curve does not show the transition temperature characteristic of a glass, but rather, a broad melting point indicative of substance possessing an ordered structure.

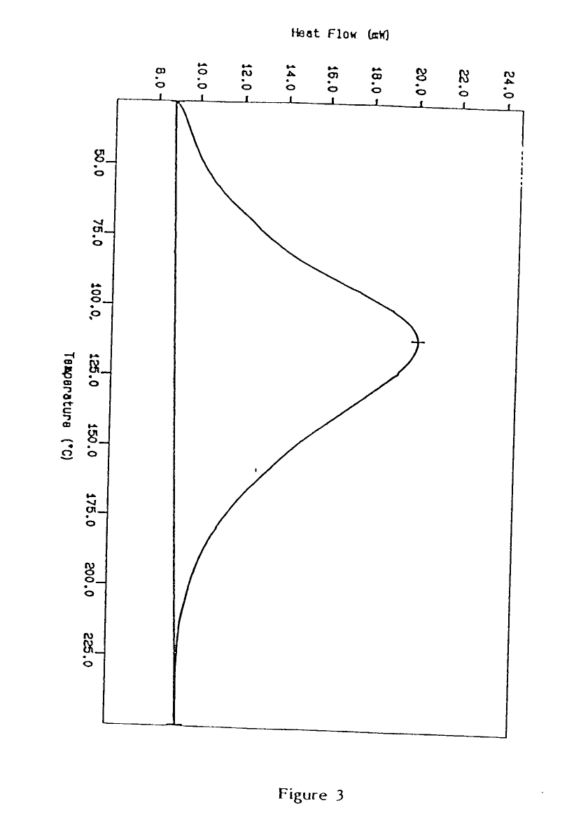

FIG. 4 depicts the DSC curve generated from a sample of pectin. As with acacia gum this curve does not show the transition temperature characteristic of a glass, but a broad melting point indicative of a substance possessing an ordered structure.

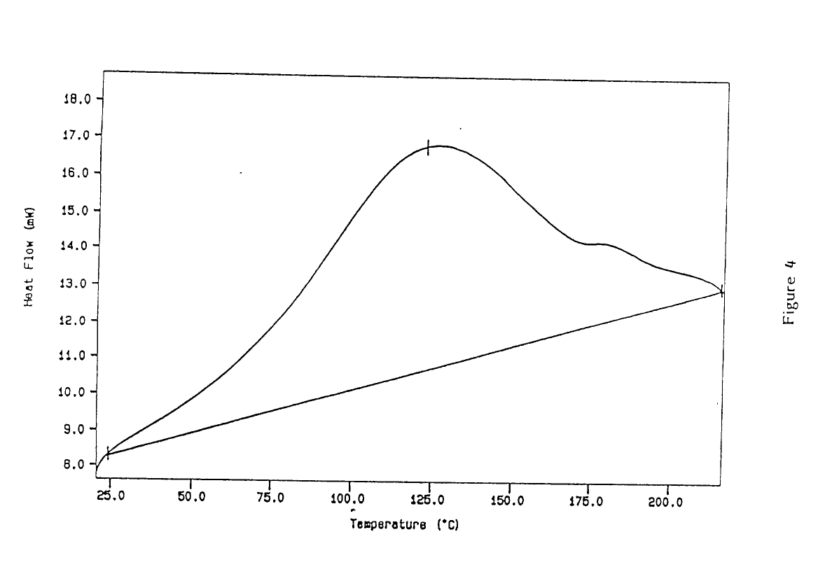

### DETAILED DESCRIPTION OF THE PREFERRED EMBODIMENTS

As previously discussed biomaterials are generally quite sensitive to environmental conditions such as pH, temperature, and/or the presence of organic or inorganic materials. These sensitivities have frustrated research efforts using biomaterials and frequently require the use of amounts in excess of those theoretically required for a particular application.

Several procedures and/or conditions have been developed in an attempt to address this problem, for example storing biomaterials in a concentrated glycerol solution at very low temperatures (−20° C. to −70° C). However, most of the currently employed procedures and/or conditions are extremely inconvenient or are very expensive.

Recent efforts have been made to improve the storage/stabilization procedures of biomaterials by immobilizing them into a solid glassy state which is stable at room temperature. As previously discussed this technique involves the dehydration of biomaterials in a glass-forming carbohydrate, particularly water soluble sugars and polymers. The stabilization of biomaterials by dehydration in a sugar matrix is also known as anhydrobiosis, and has been extensively investigated for systems involving biomembranes by J. H. Crowe and co-workers (S. Weisburd, Sci. News 133:107 (1988)). This group found that trehalose, a disaccharide, was the best and the key chemical in stabilizing biomembranes through lyophilization (Crowe, J. H. et al., J. Biochem. 242:1 (1987)). The procedure described by Crowe et al. can also be used to stabilize other biomaterials as disclosed in U.S. Pat. No. 4,891,319. Moreover, U.S. Pat. No. 5,098,893 discloses a method for stabilizing biomaterials using sucrose or ficoll (a nonionic synthetic polymer of sucrose), both of which are glass forming carbohydrates. This process is very similar to the one described in U.S. Pat. No. 4,891,319 and by Crowe et al., since trehalose is also a glass forming carbohydrate, and U.S. Pat. No. 5,098,893 teaches that the maximum protective effect of the disclosed method depends upon the vitrification of the biomaterial. Other investigators have also attributed the protective/stabilizing effect of glass forming substances like trehalose to the vitrification of the present biomaterial (Green et al., J. Phys. Chem. 93:2880-2882 (1989)). However, in the present invention, it has been surprisingly discovered that non-glass-forming substances such as acacia gum and pectin can also be used for stabilizing biomaterials. It was also unexpectedly discovered that non-glass-forming substances can protect and stabilize biomaterials better than compositions comprising glass-forming carbohydrates.

Acacia and pectin are polar/partially charged polymers (Anderson et al., J. Soc. Cosmet. Chem. 22:61-76 (1971); C. Towle Industrial Gums, R. L. Whistler, ed., Academic Press, New York, 1973), which do not form a glass. While not intended to be limiting, the stabilizing properties of acacia gum and pectin could be the result of the electrostatic interaction between the biomaterial and the polar/partially charged polymer or polyclectrolyte in solution. This interaction could form a stable polymer-biomaterial species which is maintained upon drying. The presence of acacia or pectin in solution or in the dried state has no detrimental chemical or biological interactions with the biomaterials, therefore recovery of an active biological substance can be accomplished with the addition of water or an appropriate buffer solution.

The reconstitutable dried product of the present invention can be obtained by suspending or dissolving the polar/partially charged polymer or polyelectrolyte in water or an appropriate buffer, introducing the biomaterial to be stabilized, mixing the solution, and then taking the solution to dryness. The polar/partially charged polymer or polyelectrolyte, also known as the carrier, can be any water soluble or swellable ionic or polar non-glass-forming natural or synthetic polymeric substance which does not chemically react with the biomaterial to be stored in a manner which would interfere with its perspective use, and is capable of stabilizing the present biomaterial so it can be stored in a dry water-reconstitutable state without an unacceptable loss in biological activity or function. While not intended to be limiting, examples of such polymeric substances are acacia gums, pectin, carboxymethyl cellulose, carboxymethylhydroxyethyl cellulose, guar, carboxymethyl guar, carboxymethylhydroxypropyl guar, laminaran, chitin, alginates and carrageenan.

Preferably the polar/partially changed polymer or polyelectrolyte used to obtain the composition is an acacia gum or pectin. More preferably the biomaterial is dissolved in or mixed with a solution or suspension containing approximately 0.1% to 10% (w/w) pectin or approximately 1% to 20% (w/w) acacia gum before drying. The most preferred procedure is when the biomaterial is dissolved in or mixed with a solution or suspension containing about 1% (w/w) pectin or about 7.5% (w/w) acacia gum before drying. The solution can be air dried, dried under reduced pressure or freeze-dried. Once the subject solution has been taken to dryness the resultant composition can be stored at room temperature or refrigerated whichever is most convenient and yields the best results.

Recovery of the stored biomaterial can be effected by simply adding water or an aqueous solution to an appropriate quantity of the dried composition.

The biomaterial can be isolated from the polar polymer or polyelectrolyte by any appropriate chromatographic technique, however, normally this will not be necessary since the polar polymer or polyelectrolyte should be chosen so as not to interfere with the prospective use of the stored biological substance.

For the purpose of defining this invention the term biomaterials or biological material shall mean any biologically derived substance liable to undergo a decrease in activity or function upon storage.

One category of biological materials to which the invention is applicable is proteins and peptides, including derivatives thereof such as glycoproteins. Such proteins and peptides may be enzymes, transport proteins, e.g. haemoglobin, immunoglobulins, hormones, blood clotting factors and pharmacologically active proteins or peptides.

Another category of materials to which the invention is applicable comprises nucleosides, nucleotides, dinucleotides, oligonucleotides and also enzyme cofactors. Enzyme substrates in general are also materials to which the invention may be applied.

Whole cells, membrane preparations and cell organelles can also be stabilized with the present invention.

The biological material for stabilization and storage may be isolated from a natural source, animal, plant, fungal or bacterial, or may be produced by and isolated from cells grown by fermentation in artificial culture. Such cells may or may not be genetically transformed cells.

The material will need to be soluble or suspendable in an aqueous solution or buffer, at least to the extent of forming a dilute solution or suspension which can be used to incorporate the biomaterial into the non-glass-forming carrier.

It is also possible to store more than one component of a reacting system using the present invention. This is useful for materials which may be used together in, for example, an assay or a diagnostic kit.

Storing the materials as a single preparation provides them in a convenient form for eventual use. For instance, if an assay requires the combination of a substrate, a cofactor, and an enzyme, two or all three could be stored in the composition of the invention in the required concentration ratio, and be ready for use in the assay upon reconstitution.

If multiple materials are stored, they may be mixed together in an aqueous solution and then incorporated together in the stabilizing composition. Alternatively, they may be incorporated individually into separate stabilizing compositions which are mixed together upon use.

When multiple materials are stored as a single composition, one or more of the materials may be a protein, peptide, nucleoside, nucleotide or enzyme cofactor. It is also possible that the materials may be a simpler species. For instance a standard assay procedure may require pyruvate and NADH to be present together. Both can be stored alone with acceptable stability. However, when brought together in aqueous solution they begin to react. If put together in required proportions in the stabilizing compositions of the present invention, they do not react and they can be stored for future use.

All publications mentioned in this specification are indicative of the level of skill of those skilled in the art to which this invention pertains. All publications and patents are herein incorporated by reference to the same extent as if each individual publication or patent was specifically and individually indicated to be incorporated by reference.

The above disclosure generally describes the present invention. A more complete understanding can be obtained by reference to the following specific examples which are provided herein for the purpose of illustration only and are not intended to be limiting unless otherwise specified. Unless otherwise specified all pectin and acacia solution concentrations are given as weight/weight percentages.

### EXAMPLE 1

2.0 μl of EcoR I restriction endonuclease in 50% glycerol was added to 1.0 ml of a 1.0% pectin solution containing the following buffer:

66 mM Tris acetate (pH 7.93)

20 mM MgAc₂

132 mM NaAc

6.0 mM spermidine

1.0 mM DTT

0.2 mg/ml BSA.

A 10 μl aliquot of this solution was placed into a 1.5 ml eppendorf tube and the aliquot was dried under vacuum at room temperature for 2 days.

The activity of the dried enzyme was assayed by dissolving it in 20 μl H₂O, introducing 1 μg of λ-DNA, and incubating the solution for 1 hour at 37° C. Electrophoresis of the incubated solution was then performed on a 0.8% agarose gel in TAE buffer. The results of this assay are presented in Table I and demonstrate that the enzyme retains significant activity after 4 weeks of storage at room temperature.

TABLE I

| | liquid | activity | 1 week |
|---|---|---|---|
| sample | activity | after dry | storage at RT |
| with 1% pectin | 100% | 50% | 50% |
| without pectin | 100% | 25% | 0% |

### EXAMPLE 2

6.0 μl of Hind III restriction endonuclease in 50% glycerol was added to 1.0 ml of 7.5% acacia solution containing the same buffer as in Example 1. 10 μl aliquot of this solution was placed in a 1.5 ml eppendorf tube. The aliquot was dried under vacuum at room temperature for 1 day. Activity of this enzyme was measured according to the same procedures described in Example 1. The results of this assay are presented in Table II and demonstrate that this enzyme also retains significant activity after 4 weeks of storage at room temperature.

TABLE II

| | liquid | activity | 4 week |
|---|---|---|---|
| sample | activity | after dry | RT storage |
| with 7.5% acacia | 100% | 60% | 45% |
| with 15% trehalose | 100% | 55% | 55% |

### EXAMPLE 3

(1) The Stabilization of Whole Cells with Acacia, Trehalose, and PVP.

Experiments were conducted in order to determine the relative stabilizing properties of acacia, trehalose, and polyvinylpyrrolidone (PVP) for storing whole cells. The following three stabilizing solutions were used in the experiments.

| | |
|---|---|
| solution 1 | 7.5% acacia (w/w) |
| solution 2 | 10% PVP (w/w) |
| solution 3 | 15% trehalose (w/w) |

E. coli DH10B cells were streaked from a master seed on a LB plate and grown 24 hours at 28° C. Several colonies were picked and grown overnight at 28° C. in the following medium:

1.0% Bacto Tryptone (w/v)

1.5% Bacto Yeast Extract (w/v)

10 mM NaCl

2.5 mM KCl

10 mM MgSO₄

10 mM MgCl₂.

10 ml of the overnight culture was inoculated into 1700 ml of the same medium supplemented with 0.001% polypropylene glycol (PPG-2000) (w/v) and 300 μg/ml leucine. This was incubated at 28° C. until reaching an O.D. (550 nm) of 0.4 to 0.5. The cells were then concentrated by centrifugation for 10 min. at 4000 rpm in a Sorvall RC2B centrifuge.

After centrifugation, the cells were resuspended in CCMB80 buffer which contained:

80 mM CaCl₂

20 mM MnCl₂

10 mM MgCl₂

10 mM KAc (pH 7.0)

10% glycerol,

at a cell density of 6.2×10⁹ cells/ml. Equal volume aliquots of the obtained cell solution and each stabilizing solution were mixed. 40 μl aliquots from the mixed cell-stabilizing solutions were then pipetted into the wells of a cell counting plate, and the plate shaken until the solution formed a thin film covering the bottom of each well. The solutions were dried under vacuum for 24 hours. Once dried each sample was assayed to determine the viability of the cells by first dissolving the sample in 200 μl of CCMB80 buffer at 4° C. for 15 min. A series of 10 fold diluted solutions with 0.85% NaCl were then prepared. Each diluted solution was then spread on LB plates and the cells were grown at 28° C. overnight, and viability was determined by counting colonies thereafter.

TABLE III

| Samples | Viability after Dry | |
|---|---|---|
| cells dried with acacia | 2.1 × 10⁷ | cells/ml |
| cells dried with PVP | <10⁵ | cells/ml |
| cells dried with trehalose | 10⁵ | cells/ml |

These results, shown in Table III, indicate that acacia provides greater protection for cells during the drying process than PVP or trehalose.

(2) The Effect of the Drying Method on the Stabilizing Properties of Acacia as Measured by Cell Transformation Efficiency.

DH10B cells in acacia solution are prepared and aliquoted the same as described in Example 3. The cell solutions were dried in one of the following three ways, under vacuum for 24 hours, passing a stream of dry-air over the sample for 2 hours, or by lyophilization. Once dry, the cells were reconstituted in 200 μl of CCMB80 buffer, and assayed with 50 pg pUC19 DNA for transformation according to the procedure described by Hanahan (Hanahan, D., J. Mol. Biol. 166:557 (1983)). The results of this experiment are presented in Table IV.

TABLE IV

| DH10B Cells Dried | Ratio of Transformation |
|---|---|
| in Acacia Solution | Efficiency after and before Dry |
| vacuum dried | 15% |
| air dried | 7% |
| lyophilized | 3% |

These results indicate that vacuum drying yields a higher ratio of cells capable of undergoing transformation upon reconstitution.

(3) Storage Stability of Dried DH10B Cells.

DH10B cells in acacia solution were prepared and dried under vacuum as previously described. Two additional samples of DH10B cells were lyophilized in the presence of trehalose and sucrose. All three samples were then refrigerated at −20 ° C. for 1 month. At the end of 1 month period the samples were assayed for transformation efficiency as described above, with the results presented in Table V.

TABLE V

| | remained activity | activity after |
|---|---|---|
| Samples | after dry | 1 month/−20° C. |
| cells dried in acacia | 15% | 12% |
| cells dried in trehalose | 2% | 0.4% |
| cells dried in sucrose | 2% | 0.4% |

DH10B cells when stored in pure buffer must be refrigerated at −70° C. in order to maintain a reasonable transformation efficiency. However, these results demonstrate that cells dried from an acacia solution and stored at −20° C. still retain an acceptable transformation efficiency. Moreover, these cells have a greater transformation efficiency than those stored in the presence of trehalose or sucrose, two glass-forming carbohydrates.

### EXAMPLE 4

Phycobiliprotein R-phycoerythrin was purchased from Sigma. This fluorescent red protein was blotted onto glass fiber filter paper in phosphate buffer with and without 8% w/v added acacia and dried at room temperature. Once dried, the protein was exposed to blue light to determine the amount of fluorescence. Drying in the absence of acacia caused the red protein to turn purple and lose its ability to fluoresce orange/red when illuminated with blue light. The protein sample dried in the presence of acacia retained its red color, and fluoresced upon exposure to blue light. The fluorescence of the acacia treated protein sample was as strong as a sample in solution.

### EXAMPLE 5

1.0 μl of Taq DNA polymerase in 50% glycerol was added to 100 μl of a 5% acacia solution containing the following buffer:

100 mM Tris-HCl (pH 8.4)

250 mM KCl

7.5 mM MgCl₂

1 mM dNTP mix.

A 10 μl aliquot of this solution was placed into a 0.5 ml eppendorf tube and dried under vacuum at room temperature for 2 days. Once dried, the activity of the enzyme was assayed by first dissolving it in 50 μl of H₂O, and then removing 2 μl of the aqueous solution and introducing it into 198 μl of the following assay buffer:

25 mM TAPS (pH 9.3)

50 mM KCl

2 mM MgCl₂

0.2 mM dNTP mix

1 m Ci/ml [³H] dTTP

1 mM DTT

0.5 mg/ml activated salmon sperm DNA.

This solution was incubated for 10 min at 72° C.

The incorporated deoxyribonucleotide was analyzed by counting (scintillation spectroscopy) the TCA precipitated material. The results of this assay are presented in Table VI and demonstrate that the enzyme retains significant activity after being dried.

TABLE VI

| | liquid | activity |
|---|---|---|
| sample | activity | after dry |
| with 5% acacia | 100% | 70% |
| without acacia | 100% | <10% |

Although the invention has been described and illustrated with a certain degree of particularity, it is understood that one skilled in the art will recognize a variety of additional applications and appropriate modifications within the spirit of the invention and the scope of the claims.

What is claimed is:

1. A water or aqueous buffer reconstitutable composition which is storage stable at ambient or refrigerated temperatures comprising:
i) a carrier substance which is a non-glass-forming water soluble or water swellable natural or synthetic polar/partially charged polymer or polyelectrolyte and;
ii) one or more biomaterial(s) to be stored.

2. The composition of claim 1 wherein said carrier substance is an acacia gum.

3. The composition of claim 1 wherein said carrier substance is pectin.

4. The composition of claim 1 wherein said composition is formed by the steps comprising:
i) introducing an acacia gum into a liquid at a concentration of about 7.5% (w/w);
ii) introducing one or more biomaterials to be stored, and;
iii) removing the liquid to obtain the storable composition.

5. The composition of claim 1 wherein said composition is formed by the steps comprising:
i) introducing pectin into a liquid at a concentration of about 1% (w/w);
ii) introducing one or more biomaterials to be stored, and;
iii) removing the liquid to obtain the storable composition.

6. The composition of claim 1 wherein the biomaterial to be stored is selected from the group consisting of proteins, peptides, nucleosides, nucleotides, dimers or oligomers of nucleosides or nucleotides, enzyme cofactors, biological membranes, cells, cell organelles and derivatives of the foregoing.

7. A method of rendering a biomaterial storage stable at ambient or refrigerated temperatures comprising:
i) introducing a carrier substance which is a non-glass-forming water soluble or water swellable natural or synthetic polar/partially charged polymer or polyelectrolyte into water or an appropriate buffer and;
ii) adding one or more biomaterial(s) to be stored;
iii) mixing the resulting suspension or solution and;
iv) removing the water to form the water or buffer reconstitutable composition.

8. The method of claim 7 wherein the water is removed by evaporation under subatmospheric pressure.

9. The method of claim 7 wherein the water is removed by blowing a stream of dry gas over the surface of the suspension or solution.

10. The method of claim 7 wherein the water is removed by lyophilization.

11. The method of claim 7 wherein said polar/partially charged polymer or polyelectrolyte is an acacia gum.

12. The method of claim 7 wherein said polar/partially charged polymer or polyelectrolyte is pectin.

13. The method of claim 11 wherein said acacia gum is added to the water or buffer at a concentration of about 7.5% (w/w).

14. The method of claim 12 wherein said pectin is added to the water or buffer at a concentration of about 1% (w/w).
